(19) United States
(12) Patent Application Publication
(10) Pub. No.: US 2024/0165023 A1
(43) Pub. Date: May 23, 2024

(54) LIQUID PHARMACEUTICAL COMPOSITION

(71) Applicant: Verona Pharma PLC, Cardiff (GB)

(72) Inventors: Peter Lionel Spargo, Canterbury (GB); Tara Renae Rheault, Cary, NC (US); Doris K. Weilert, Lee's Summit, MO (US)

(21) Appl. No.: 18/417,410

(22) Filed: Jan. 19, 2024

Related U.S. Application Data
(63) Continuation of application No. PCT/GB2023/052085, filed on Aug. 7, 2023.
(60) Provisional application No. 63/417,762, filed on Oct. 20, 2022.
(60) Provisional application No. 63/370,694, filed on Aug. 8, 2022.
(60) Provisional application No. 63/370,696, filed on Aug. 8, 2022.
(60) Provisional application No. 63/370,699, filed on Aug. 8, 2022.
(60) Provisional application No. 63/502,977, filed on May 18, 2023.

(51) Int. Cl. A61K 9/00; A61K 9/08; A61K 31/519; A61K 47/02; A61K 47/26; A61P 11/08

(52) U.S. Cl. CPC A61K 9/0078; A61K 9/08; A61K 31/519; A61K 47/02; A61K 47/26; A61P 11/08

(57) ABSTRACT

The invention relates to a liquid pharmaceutical composition comprising a dose of a compound which is ensifentrine or a pharmaceutically acceptable salt thereof, wherein the liquid pharmaceutical composition provides a blood plasma concentration of ensifentrine after administration by inhalation to a human subject having COPD, which blood plasma concentration of ensifentrine has: a mean Cₘₐₓ of from about 400 pg/mL to about 720 pg/mL; and/or a mean AUC₀₋ₜₐᵤ of from about 2000 pg/mL*h to about 3000 pg/mL*h; and/or a mean Tₘₐₓ at from about 0.6 hours to about 1.5 hours. The invention also provides a method of treating COPD.

### CROSS REFERENCE

This application is a continuation of International Patent Application No. PCT/GB2023/052085, filed Aug. 7, 2023, which claims the benefit of U.S. Provisional Application Nos. 63/370,694, filed Aug. 8, 2022, 63/370,696, filed Aug. 8, 2022, 63/370,699, filed Aug. 8, 2022, and 63/502,977, filed May 18, 2023, each of which are incorporated herein by reference in their entirety.

### FIELD OF THE INVENTION

The present invention relates to a liquid pharmaceutical composition comprising a dose of a compound which is ensifentrine or a pharmaceutically acceptable salt thereof.

### BACKGROUND OF THE INVENTION

Ensifentrine (N-(2-{(2E)-9,10-dimethoxy-4-oxo-2-[(2,4,6-trimethylphenyl)imino]-6,7-dihydro-2H-pyrimido[6,1-a]isoquinolin-3(4H)-yl}ethyl)urea; also known as RPL554) is a dual PDE3/PDE4 inhibitor and is described in WO 00/58308 A1.

As a combined PDE3/PDE4 inhibitor, ensifentrine has both bronchodilatory and anti-inflammatory activity and is useful in the treatment of respiratory disorders including chronic obstructive pulmonary disease (COPD). The chemical structure of ensifentrine is shown below.

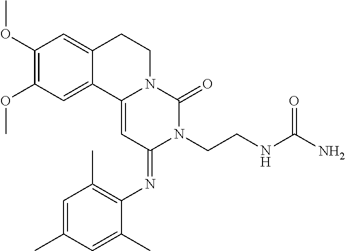 [SMILES: COc1cc2c(cc1OC)-c1c/c(=N\c3c(C)cc(C)cc3C)n(CCNC(N)=O)c(=O)n1CC2]

COPD is a progressive, long-term condition. Ensifentrine is typically administered to COPD patients by nebuliser as a maintenance therapy. It would be beneficial to provide a blood plasma concentration of ensifentrine having a desirable pharmacokinetic profile in COPD patients following inhalation of a liquid pharmaceutical composition suitable for inhalation.

### SUMMARY OF THE INVENTION

It has been found that a liquid pharmaceutical composition comprising a dose of ensifentrine or a pharmaceutically acceptable salt thereof can provide an advantageous blood plasma concentration of ensifentrine following inhalation to a human subject having COPD.

The invention accordingly provides a liquid pharmaceutical composition comprising a dose of a compound which is ensifentrine or a pharmaceutically acceptable salt thereof, wherein the liquid pharmaceutical composition provides a blood plasma concentration of ensifentrine after administration by inhalation to a human subject having COPD, which blood plasma concentration of ensifentrine has:
a mean Cₘₐₓ of from about 400 pg/mL to about 720 pg/mL; and/or
a mean AUC₀₋ₜₐᵤ of from about 2000 pg/mL*h to about 3000 pg/mL*h; and/or
a mean Tₘₐₓ at from about 0.6 hours to about 1.5 hours.

The invention also provides an ampule comprising the liquid pharmaceutical composition. Further provided by the invention is a nebuliser comprising the liquid pharmaceutical composition.

Also provided by the invention is a method of treating COPD in a human subject, the method comprising administering the liquid pharmaceutical composition to the human subject be inhalation.

### DETAILED DESCRIPTION OF THE INVENTION

The liquid pharmaceutical composition provides a blood plasma concentration of ensifentrine after administration by inhalation to a human subject having COPD, which blood plasma concentration of ensifentrine has: a mean Cₘₐₓ of from about 400 pg/mL to about 720 pg/mL; and/or a mean AUC₀₋ₜₐᵤ of from about 2000 pg/mL*h to about 3000 pg/mL*h; and/or a mean Tₘₐₓ at from about 0.6 hours to about 1.5 hours. As such, the liquid pharmaceutical composition is suitable for providing the blood plasma concentration of ensifentrine after administration by inhalation to a human subject having COPD, for instance after administration by inhalation from a nebuliser.

Cₘₐₓ, AUC₀₋ₜₐᵤ and Tₘₐₓ are pharmacokinetic parameters well known to the skilled person. Cₘₐₓ is the maximum concentration of the compound in the blood plasma achieved following administration of the compound. Tₘₐₓ is the time at which Cₘₐₓ observed. AUC is the area under the curve of the blood plasma concentration of the compound as a function of time following administration over a given period. AUC₀₋ₜₐᵤ is the AUC from administration to the end of the dosing period (which may for instance be 8 hours, 12 hours or 24 hours). As used herein, tau is typically 12 hours and AUC₀₋ₜₐᵤ is AUC₀₋₁₂ₕ.

Typically, the liquid pharmaceutical composition provides a blood plasma concentration of ensifentrine having a mean Cₘₐₓ of from about 400 pg/mL to about 720 pg/mL after administration by inhalation to a human subject having COPD. The mean Cₘₐₓ may be from about 500 pg/mL to about 600 pg/mL. For instance, the mean Cₘₐₓ may be from about 500 pg/mL to about 550 pg/mL, or from about 520 pg/mL to about 525 pg/mL.

Typically, the liquid pharmaceutical composition provides a blood plasma concentration of ensifentrine having a mean AUC₀₋ₜₐᵤ of from about 2000 pg/mL*h to about 3000 pg/mL*h after administration by inhalation to a human subject having COPD. The mean AUC₀₋ₜₐᵤ may be from about 2300 pg/mL*h to about 2600 pg/mL*h. For instance, the mean AUC₀₋ₜₐᵤ may be from about 2400 pg/mL*h to about 2500 pg/mL*h, or from about 2425 pg/mL*h to about 2475 pg/mL*h.

The liquid pharmaceutical composition may provide a blood plasma concentration of ensifentrine having a mean Cₘₐₓ of from about 400 pg/mL to about 720 pg/mL and a mean AUC₀₋ₜₐᵤ of from about 2000 pg/mL*h to about 3000 pg/mL*h after administration by inhalation to a human subject having COPD.

Typically, the liquid pharmaceutical composition provides a blood plasma concentration of ensifentrine having a mean Tₘₐₓ at from about 0.6 hours to about 1.5 hours after inhalation by a human subject having COPD. The mean Tₘₐₓ may be at from about 0.8 hours to about 1.3 hours. For instance, the mean Tₘₐₓ may be at from about 0.9 hours to about 1.1 hours, about 0.95 hours to about 1.05 hours.

The liquid pharmaceutical composition may provide a blood plasma concentration of ensifentrine after administration by inhalation to a human subject having COPD, which blood plasma concentration of ensifentrine has: a mean Cₘₐₓ of from about 500 pg/mL to about 600 pg/mL; and a mean AUC₀₋ₜₐᵤ of from about 2300 pg/mL*h to about 2600 pg/mL*h; and a mean Tₘₐₓ at from about 0.8 hours to about 1.3 hours. The blood plasma concentration of ensifentrine may have: a mean Cₘₐₓ of from about 510 pg/mL to about 530 pg/mL; and a mean AUC₀₋ₜₐᵤ of from about 2350 pg/mL*h to about 3550 pg/mL*h; and a mean Tₘₐₓ at from about 0.9 hours to about 1.1 hours.

As used herein, the term “about” may represent a variation of ±10% of the stated value.

The mean characteristics of the blood plasma concentration provided in human subjects with COPD may be determined based on the observed mean blood plasma concentration of ensifentrine following administration of the liquid pharmaceutical composition by inhalation to a sample of human subjects having COPD. Typically, the mean Cₘₐₓ, mean AUC₀₋ₜₐᵤ and mean Tₘₐₓ are as measured by determining the blood plasma concentration in a sample of human subjects having COPD at intervals after administration of the liquid pharmaceutical composition. The sample of human subjects may comprise from 100 to 1000 human subjects, for instance 300 human subjects. The human subjects in the sample may have ages in the range of 45 to 75 years.

The skilled person is aware of the suitable intervals for measuring blood plasma concentration following inhalation of a liquid pharmaceutical composition. Typically, the intervals are at two or more of −0.5 hours (±0.5 h, i.e. pre-dose), 0.5 h (±0.25 h), 1.0 h (±0.5 h), 1.5 h (±0.5 h), 2.0 h (±0.5 h) 2.5 h (±0.5 h), 3.0 h (±0.5 h), 4.0 h (±1 h), 6.0 h (±h), 8.0 h (±h), 10.0 h (±h), 12.0 h (±h), 24.0 h (±h), 36.0 h (±h), 48.0 h (±h), 56.0 h (±h) and 60.0 h (±h) after administration of the liquid pharmaceutical composition. The intervals may be at two or more of −0.5 hours (±0.5 h, i.e. pre-dose), 1.0 h (±0.5 h), 2.0 h (±0.5 h), 3.0 h (±0.5 h), 4.0 h (±h), 12.0 h (±h), 24.0 h (±h), 36.0 h (±1 h), 48.0 h (±1 h), and 56.0 h (±1 h) after administration of the liquid pharmaceutical composition. For instance, the intervals may be at −0.5 hours (±0.5 h, i.e. pre-dose), 0.5 h (±0.25 h), 1.0 h (±0.5 h), 2.0 h (±0.5 h), 3.0 h (±0.5 h), 4.0 h (±1 h) and 12.0 h (±h).

Typically, the blood plasma concentration is determined using an analytical method having an LLOQ of no greater than about 5.0 pg/mL.

Administration of the liquid pharmaceutical composition by inhalation to a human subject typically comprises administration of the liquid pharmaceutical composition to the human subject by inhalation from a nebuliser. Nebulisers aerosolise a liquid pharmaceutical composition into an aerosol that is inhaled into a human subject's respiratory tract. Examples of nebulisers include a soft mist nebuliser, a vibrating mesh nebuliser, a jet nebuliser and an ultrasonic wave nebuliser. Suitable nebuliser devices include the Philips I-Neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebuliser may for instance be a PARI LC Sprint jet nebuliser with a PARI Vios® PRO Aerosol Delivery System PARI BOY® compressor. The liquid pharmaceutical composition may be inhaled via the nebuliser for from 1 to 15 minutes, for instance from 5 to 10 minutes.

For instance, the mean Cₘₐₓ, mean AUC₀₋ₜₐᵤ and mean Tₘₐₓ may be as measured by determining the mean blood plasma concentration in a sample of 300 human subjects having COPD at intervals after administration of the liquid pharmaceutical composition, wherein the human subjects in the sample may have ages in the range of 45 to 75 years and wherein the liquid pharmaceutical composition may be inhaled via the nebuliser for from 1 to 15 minutes (for instance about 7 minutes). The human subjects may have moderate COPD.

The human subject is typically receiving the compound as a maintenance therapy. The compound may be administered to the human subject once, twice or three times daily. The compound is preferably administered as a twice-daily maintenance therapy.

The liquid pharmaceutical composition comprises a dose of a compound which is ensifentrine or a pharmaceutically acceptable salt thereof. Typically, the dose is from about 2 mg to about 4 mg of the compound. For instance, the dose may be from 2.5 mg to 3.5 mg of the compound. The dose may be from 2.8 mg to 3.2 mg of the compound. The dose is typically about 3 mg of the compound, for example about 3.0 mg.

The dose may be from 2.8 mg to 3.2 mg of ensifentrine (i.e. ensifentrine free base). The dose is typically about 3 mg of ensifentrine, for example 3.0 mg of ensifentrine.

The concentration of the compound in the liquid pharmaceutical composition is typically from 0.8 to 1.6 mg/mL. For instance, the liquid pharmaceutical composition typically comprises ensifentrine at a concentration of from 1.0 to 1.4 mg/mL. The liquid pharmaceutical composition may comprise ensifentrine at a concentration of from 1.1 to 1.3 mg/mL. The liquid pharmaceutical composition may comprise about 1.2 mg/mL ensifentrine.

Inhalation of the liquid pharmaceutical composition typically comprises inhalation only of a portion of the liquid pharmaceutical composition. As such, only a portion of the liquid pharmaceutical composition is typically delivered to the patient, for instance due to a remainder of the liquid pharmaceutical composition remaining in a nebuliser used to deliver the liquid pharmaceutical composition by inhalation. The portion of the liquid pharmaceutical composition delivered to the lungs of the patient from a nebuliser may be from 20 to 40% by volume of the liquid pharmaceutical composition originally present in the nebuliser prior to administration. The portion of the liquid pharmaceutical composition delivered to the lungs of the patient from a nebuliser may be from 25 to 35% by volume of the liquid pharmaceutical composition originally present in the nebuliser prior to administration, for instance from 29 to 33% by volume or about 31% by volume. As such the mean delivered dose of the compound inhaled by the human subject is typically from 20 to 40%, from 25 to 35%, or from 29 to 33% of the overall dose present in the liquid pharmaceutical composition prior to administration. The mean delivered dose of the compound (e.g. the mean delivered dose of ensifentrine free base) is typically from 0.7 to 1.1 mg when administered using a nebuliser. The mean delivered dose may be from 0.8 to 1.0 mg, for instance from 0.90 to 0.95 mg. The nebuliser is typically a jet nebuliser. The liquid pharmaceutical composition is typically suitable for providing the mean Cₘₐₓ, mean AUC₀₋ₜₐᵤ and mean Tₘₐₓ defined herein following delivery from a nebulizer as described above.

The invention also provides a liquid pharmaceutical composition as defined herein, which liquid pharmaceutical composition provides a mean delivered dose of from 0.7 to 1.1 mg of the compound when administered using a nebuliser. The mean delivered dose may be from 0.8 to 1.0 mg, for instance from 0.90 to 0.95 mg.

The liquid pharmaceutical composition typically provides a mean increase in baseline FEV₁ of at least 20 mL after administration by inhalation to the human subject. For instance, the liquid pharmaceutical composition may provide a mean increase in baseline FEV₁ of at least 30 mL after administration by inhalation to the human subject. Typically, as used herein, FEV₁ is determined as set out in the article Standardisation of Spirometry, Eur J 2005; 26; 319-338.

The liquid pharmaceutical composition typically comprises (a) a suspension of particles comprising the compound and (b) a diluent. The diluent is typically water. It may be the case that some or all of the particles comprising the compound in the liquid pharmaceutical composition have settled to the bottom of a receptacle containing the liquid pharmaceutical composition, for instance after storage for a period of time. The particles comprising the compound may be re-suspended in any suitable way, for instance by agitation of the sterile liquid pharmaceutical composition.

The particles comprising the compound typically have a Dv50 of from about 0.2 μm to about 5.0 μm. For instance, the particles comprising the compound may have a Dv50 of from about 1.0 μm to about 2.2 μm, or from about 1.1 μm to about 1.5 μm.

Particle sizes are described herein by reference to the Dv50 value, which is the median particle size for a volume distribution. Thus, half the volume of the particles have diameters of less than the Dv50 value and half the volume of the particles have diameters of greater than the Dv50 value. This is a well-known manner in which to describe particle size distributions. The parameters of Dv10 and Dv90 may also be used to characterise a particle size distribution of a sample. 10% of the volume of particles have a diameter of less than the Dv10 value. 90% of the volume of the particles have a diameter of less than the Dv90 value.

The particles comprising the compound typically have a Dv10 of from about 0.3 μm to about 0.9 μm and/or a Dv90 of from about 2.3 μm to about 4.5 μm.

For instance, the particles comprising the compound may have: a Dv10 of from about 0.3 μm to about 0.9 μm; and a Dv50 of from about 1.0 μm to about 2.2 μm; and a Dv90 of from about 2.3 μm to about 4.5 μm.

The technique used to measure the Dv10, Dv50 and Dv90 values as stated herein is typically laser diffraction. The particle size distribution of the particles comprising the compound may be as measured by laser diffraction using a wet powder dispersion system. For instance, the particle size distribution can be measured by laser diffraction using a Malvern Spraytec in conjunction with a wet dispersion cell. Typically, the instrument parameters for the Malvern Spraytec are as follows:
particle—standard opaque particle;
refractive index Particle—1.50;
refractive index (imaginary)—0.50;
density of particle—1.00;
refractive index of dispersant—1.33;
controller unit—1000 RPM;
measurement type—timed;
initial sampling time—30 s;
obscuration—20%-30%;
dispersant—1% Polysorbate 20 in deionised water.

The ensifentrine particles may be produced by any pharmaceutically acceptable size reduction process or particle size controlled production process. For instance, the particles may be produced by spray-drying a solution of ensifentrine, by controlled crystallisation, or by size reduction of a solid form of ensifentrine, for example by air jet milling, mechanical micronisation or media milling.

The particles comprising the compound typically comprise ensifentrine (i.e. ensifentrine free base). The particles comprising the compound may comprise at least 90 wt % ensifentrine free base relative to the total weight of the particles, for instance at least 95 wt % ensifentrine. The particles may comprise at least 99 wt % ensifentrine. The particles may consist of ensifentrine.

The compound is typically in crystalline form. The particles comprising the compound typically comprise at least 90 wt % ensifentrine free base Form I relative to the total weight of the particles. Ensifentrine free base Form I is a crystalline polymorph of ensifentrine (crystalline polymorph Form 1) which typically has a powder X-ray diffraction pattern comprising characteristic peaks at 10.1° and 12.9°±0.1° 2θ. As stated herein, values of °2θ are typically as measured using an X-ray wavelength of CuKα radiation (λ=1.5406 Å). The powder X-ray diffraction pattern of Form I typically further comprises characteristics peaks at 15.3° and 17.6°±0.1° 2θ. Form I of ensifentrine may have an powder X-ray diffraction pattern comprising at least 5 characteristic peaks selected from 6.4°, 10.1°, 12.6°, 12.9°, 13.6°, 14.2°, 14.7°, 15.3°, 15.4°, 15.8°, 17.0°, 17.6°, 18.9°, 20.9°, 22.4°, 22.8° and 28.7°±0.1° 2θ. Crystalline polymorph Form I typically has a differential scanning calorimetry trace showing a maximum at 248° C.

The particles comprising the compound typically comprise at least 95 wt % or at least 99 wt % of ensifentrine crystalline polymorph Form I relative to the total weight of the particles. The particles of the compound may consist essentially of ensifentrine crystalline polymorph Form 1.

The liquid pharmaceutical composition typically comprises, relative to the total weight of the liquid pharmaceutical composition:
(i) ensifentrine particles at a concentration of from 0.8 to 1.6 mg/mL, which ensifentrine particles have a Dv50 of from about 1.0 μm to about 2.2 μm and optionally have a Dv10 of from about 0.3 μm to about 0.9 μm and a Dv90 of from about 2.3 μm to about 4.5 μm;
(ii) one or more surfactants at a total concentration of from 0.1 to 1.0 mg/mL;
(iii) one or more buffers at a total concentration of from 1.0 to 2.0 mg/ml; and (iv) water.

The liquid pharmaceutical composition may optionally further comprise (v) a tonicity adjuster at a concentration from 6.0 to 12.0 mg/mL. The tonicity adjuster is typically sodium chloride.

The liquid pharmaceutical composition may comprise, relative to the total weight of the liquid pharmaceutical composition: (i) ensifentrine particles at a concentration of from 1.0 to 1.4 mg/mL, which ensifentrine particles have a Dv50 of from about 1.0 μm to about 2.2 μm and optionally have a Dv10 of from about 0.3 μm to about 0.9 μm and a Dv90 of from about 2.3 μm to about 4.5 μm; (ii) one or more surfactants at a total concentration of from 0.4 to 0.7 mg/mL; (iii) one or more buffers at a total concentration of from 1.4 to 1.8 mg/ml; (iv) water; and a tonicity adjuster at a concentration from 7.0 to 10.0 mg/mL.

The liquid pharmaceutical composition may comprise at least 95 wt % or at least 99 wt % of (i), (ii), (iii), (iv) and optionally (v) relative to the total weight of the liquid pharmaceutical composition.

Examples of buffers include a citrate buffer, a phosphate buffer, an acetate buffer, and a bicarbonate buffer. Preferably, the one or more buffers comprise a phosphate buffer, for instance sodium dihydrogen phosphate dihydrate and/or disodium phosphate dihydrate.

Examples of surfactants include lecithin, oleic acid, polyoxyethylene glycol alkyl ethers (for instance PEG 300, PEG 600, PEG 1000, Brij 30, Brij 35, Brij 56, Brij 76 and Brij 97), polypropylene glycol (for instance PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (polysorbates, for instance polysorbate 20, polysorbate 40, polysorbate 60 and polysorbate 80), sorbitan alkyl esters (for instance sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (for instance Pluronic surfactants), polyvinyl pyrrolidone K25, polyvinyl alcohol, oligolactic acid, sodium dioctyl sulfosuccinate and polyethoxylated tallow amine (POEA).

Preferably, the one or more surfactants comprise a polysorbate and/or a sorbitan alkyl ester. The one or more surfactants may for instance comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). The one or more surfactants may for instance comprise sorbitan monolaurate (Span 20), sorbitan monooleate (Span 80) or sorbitan trioleate (Span 85). Preferably, the one or more buffers comprise polysorbate 20 (Tween 20) and/or sorbitan monolaurate (Span 20).

The liquid pharmaceutical composition may comprise, relative to the total weight of the liquid pharmaceutical composition: (i) ensifentrine particles at a concentration of from 1.0 to 1.4 mg/mL, which ensifentrine particles have a Dv50 of from about 1.0 μm to about 2.2 μm and optionally have a Dv10 of from about 0.3 μm to about 0.9 μm and a Dv90 of from about 2.3 μm to about 4.5 μm; (ii) one or more surfactants at a total concentration of from 0.4 to 0.7 mg/mL, which one or more surfactants are selected from a polysorbate and/or a sorbitan alkyl ester; (iii) one or more buffers at a total concentration of from 1.4 to 1.8 mg/mL, which one or more buffers are selected from phosphate buffers; (iv) water; and sodium at a concentration from 7.0 to 10.0 mg/mL.

The ensifentrine particles are particles comprising ensifentrine free base. The ensifentrine particles typically comprise at least 90.0 wt % of ensifentrine, preferably at least 95.0 wt %. The ensifentrine particles may consist essentially of ensifentrine, or may consist of ensifentrine. The ensifentrine particles typically comprise at least 90.0 wt % or at least 95.0 wt % of crystalline polymorph Form I of ensifentrine as defined herein. For instance, the ensifentrine particles may comprise at least 90 wt % of ensifentrine in the form of crystalline polymorph Form I having an powder X-ray diffraction pattern comprising at least 5 characteristic peaks selected from 6.4°, 10.1°, 12.6°, 12.9°, 13.6°, 14.2°, 14.7°, 15.3°, 15.4°, 15.8°, 17.0°, 17.6°, 18.9°, 20.9°, 22.4°, 22.8° and 28.7°±0.1° 2θ.

The liquid pharmaceutical composition may comprise, relative to the total weight of the liquid pharmaceutical composition:
ensifentrine particles at a concentration of from 1.0 to 1.4 mg/mL, which ensifentrine particles have a Dv10 of from about 0.3 μm to about 0.9 μm, a Dv50 of from about 1.0 μm to about 2.2 μm and a Dv90 of from about 2.3 μm to about 4.5 μm;
polysorbate 20 (Tween 20) at a concentration of 0.3 to 0.7 mg/mL;
sorbitan monolaurate (Span 20) at a concentration of 0.0 to 0.1 mg/mL;
sodium dihydrogen phosphate dihydrate at a concentration of 0.5 to 1.0 mg/mL;
disodium hydrogen phosphate dihydrate at a concentration of 0.5 to 1.0 mg/mL;
sodium chloride at a concentration of 5 to 10 mg/mL; and
water, and
   wherein the total weight of ensifentrine in the liquid pharmaceutical composition is from 2.7 to 3.3 mg. The liquid pharmaceutical composition may comprise, relative to the total weight of the liquid pharmaceutical composition, at least 95 wt % or at least 99 wt % of the listed components.

The liquid pharmaceutical composition may comprise, relative to the total weight of the liquid pharmaceutical composition:
ensifentrine particles at a concentration of from 1.1 to 1.3 mg/mL, which ensifentrine particles have a Dv10 of from 0.4 μm to 0.7 μm, a Dv50 of from 1.2 μm to 1.9 μm and a Dv90 of from 2.9 μm to 4.3 μm;
polysorbate 20 (Tween 20) at a concentration of 0.4 to 0.6 mg/mL;
sorbitan monolaurate (Span 20) at a concentration of 0.02 to 0.08 mg/mL;
sodium dihydrogen phosphate dihydrate at a concentration of 0.65 to 0.85 mg/mL;
disodium hydrogen phosphate dihydrate at a concentration of 0.75 to 0.95 mg/mL;
sodium chloride at a concentration of 7 to 9 mg/mL; and
water, and
   wherein the total weight of ensifentrine in the liquid pharmaceutical composition is from 2.8 to 3.2 mg, and wherein the ensifentrine particles comprise at least 95 wt % ensifentrine crystalline polymorph Form I relative to the total weigh of the ensifentrine particles. The liquid pharmaceutical composition may comprise, relative to the total weight of the liquid pharmaceutical composition, at least 95 wt % or at least 99 wt % of the listed components.

The liquid pharmaceutical composition may comprise, relative to the total weight of the liquid pharmaceutical composition:
ensifentrine particles at a concentration of about 1.2 mg/mL, which ensifentrine particles have a Dv10 of from 0.4 μm to 0.7 μm, a Dv50 of from 1.2 μm to 1.9 μm and a Dv90 of from 2.9 μm to 4.3 μm;
polysorbate 20 (Tween 20) at a concentration of about 0.5 mg/mL;
sorbitan monolaurate (Span 20) at a concentration of about 0.05 mg/mL;
sodium dihydrogen phosphate dihydrate at a concentration of about 0.74 mg/mL;
disodium hydrogen phosphate dihydrate at a concentration of about 0.85 mg/mL;
sodium chloride at a concentration of about 8.6 mg/mL; and
water, and
   wherein the total weight of ensifentrine in the liquid pharmaceutical composition is about 3.0 mg.

Typically, the total volume of the liquid pharmaceutical composition is from 2.0 to 3.0 mL. For instance, the total volume of the liquid pharmaceutical composition may be about 2.5 mL.

The invention also provides a liquid pharmaceutical composition comprising, relative to the total weight of the liquid pharmaceutical composition:
ensifentrine particles at a concentration of from 1.0 to 1.4 mg/mL, which ensifentrine particles have a Dv10 of from about 0.3 μm to about 0.9 μm, a Dv50 of from about 1.0 μm to about 2.2 μm and a Dv90 of from about 2.3 μm to about 4.5 μm;
polysorbate 20 (Tween 20) at a concentration of 0.3 to 0.7 mg/mL;
sorbitan monolaurate (Span 20) at a concentration of 0.0 to 0.1 mg/mL;
sodium dihydrogen phosphate dihydrate at a concentration of 0.5 to 1.0 mg/mL;
disodium hydrogen phosphate dihydrate at a concentration of 0.5 to 1.0 mg/mL;
sodium chloride at a concentration of 5 to 10 mg/mL; and
water, and
   wherein the total weight of ensifentrine in the liquid pharmaceutical composition is from 2.7 to 3.3 mg. The liquid pharmaceutical composition may be as further defined herein.

The ampule typically comprises from 2.0 to 3.0 mL of the liquid pharmaceutical composition. The nebuliser comprises the liquid pharmaceutical composition, and the liquid pharmaceutical composition is typically comprised in an ampule. The nebuliser may be a soft mist nebuliser, a vibrating mesh nebuliser, a jet nebuliser or an ultrasonic wave nebuliser. The nebuliser is typically a jet nebuliser. The nebuliser may for instance be a PARI LC Sprint jet nebulizer with a PARI Vios® PRO Aerosol Delivery System PARI BOY® compressor.

The method of treating COPD in a human subject comprises administering the liquid pharmaceutical composition to the human subject by inhalation. Typically, a therapeutically effective amount of the liquid pharmaceutical composition is administered to the human subject by inhalation.

The COPD is typically moderate COPD or severe COPD. The above stages of COPD can be classified as set out below, where FEV₁ is forced expiratory volume in 1 second and FVC is forced vital capacity.
Mild COPD: FEV₁/FVC<0.7 and FEV₁ 80% predicted
Moderate COPD: FEV₁/FVC<0.7 and 50% 5 FEV₁<80% predicted
Severe COPD: FEV₁/FVC<0.7 and 30% 5 FEV₁<50% predicted
Very Severe COPD: FEV₁/FVC<0.7 and FEV₁<30% predicted

In each case the actual FEV₁ for the human subject is compared with a predicted FEV₁ value based on factors such as age and height of the human subject. These predicted values are readily available to the skilled person, for instance from the National Health and Nutrition Examination Survey Ill (Hankinson J L, Odencrantz J R, Fedan K B. Spirometry reference values from a sample of the general U.S. Population. Am J Respir Crit Care. 1999; 159:179-187). Examples of equations for calculating the predicted FEV₁ (in L) for a human subject are as follows, where H is height (cm) and A is age (yrs):
Males: 0.0430H-0.0290A-2.490
Females: 0.0395H-0.025A-2.600

The FEV₁ and FVC used to determine the severity of COPD in a human subject are measured by carrying out spirometry shortly after the administration of an adequate dose of at least one short-acting inhaled bronchodilator. Typically, measurement of FEV₁ and FVC for determining COPD disease severity is done between 15 and 30 minutes following administration of salbutamol (albuterol).

Typically, as used herein, FEV₁ and FVC are determined as set out in the article Standardisation of spirometry, Eur J 2005; 26; 319-338.

The human subject may have been determined to have moderate COPD by measuring FEV₁/FVC<0.7 and 50% 5 FEV₁<80% predicted FEV₁ value, where FEV₁ is forced expiratory volume in 1 second and FVC is forced vital capacity as measured between 15 and 30 minutes after a dose of a bronchodilator, optionally wherein the bronchodilator is salbutamol. The human subject may have been determined to have severe COPD by measuring FEV₁/FVC<0.7 and 30% 5 FEV₁<50% predicted, where FEV₁ is forced expiratory volume in 1 second and FVC is forced vital capacity as measured between 15 and 30 minutes after a dose of a bronchodilator, optionally wherein the bronchodilator is salbutamol. The determination of the human subject's COPD severity may take place at least 1 day prior to the first administration of the compound.

The human subject may be male. The human subject may be female. The human subject may have an age of greater than or equal to 65 years. The human subject may have an age of less than 65 years. The human subject may be taking a background medication selected from one or more of a long-acting muscarinic antagonist (LAMA), a long-acting beta-agonist (LABA) and an inhaled corticosteroid (ICS). In some cases, the human subject is not receiving a background medication. For instance, the human subject may not be taking a background medication, which background medication is a long-acting muscarinic antagonist (LAMA), a long-acting beta-agonist (LABA) or an inhaled corticosteroid (ICS).

Preferably, the method comprises administering the compound to the human subject by inhalation from a nebuliser. Nebulisers aerosolise a liquid pharmaceutical composition into an aerosol that is inhaled into a human subject's respiratory tract. Examples of nebulisers include a soft mist nebuliser, a vibrating mesh nebuliser, a jet nebuliser and an ultrasonic wave nebuliser. Suitable nebuliser devices include the Philips I-Neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebuliser may for instance be a PARI LC Sprint jet nebulizer with a PARI Vios® PRO Aerosol Delivery System PARI BOY® compressor. The compound may be inhaled via the nebuliser for from 1 to 15 minutes, for instance for from 5 to 10 minutes, or around 7 minutes.

Typically, the method comprises administering the compound to the human subject once, twice or three times per day, for instance twice or three times per day. The compound may be administered to the human subject by inhalation once, twice or three times a day. Preferably the method comprises administering the compound to the human subject by inhalation twice a day. The method may comprise administering a first dose of the compound in the morning (for instance within 3 hours following waking) and a second dose of the compound in the evening (for instance within 3 hours before bed). Typically, the morning and evening doses are administered from 10 to 14 hours apart, for instance about 12 hours apart.

Typically, the compound is administered twice a day in two separate doses which are the same or similar. Typically, the method may comprise administering the compound to the human subject twice a day in a first dose of from 2 to 4 mg and a second dose of from 2 to 4 mg.

Preferably, the method comprises administering two doses of the liquid pharmaceutical composition comprising about 3 mg ensifentrine free base to the human subject per day by inhalation. The method preferably comprises administering a dose of about 3 mg of the compound to the human subject twice a day (3 mg BID) by inhalation. More preferably, the method comprises administering by nebuliser a dose of about 3 mg the compound to the human subject twice a day. Each dose may be 3.0 mg free base ensifentrine administered by nebulizer.

The compound is typically used as a maintenance therapy. Typically, the method comprises administering the compound to the human subject at least once per day for at least 8 weeks. The compound may be administered to the human subject at least once per day for at least 16 weeks, preferably for at least 24 weeks. The compound may be administered daily to the human subject for at least 1 year. The method may comprise administering the compound to the human subject at least once every 24 hours, preferably at least twice every 24 hours, for at least 8 weeks, preferably for at least 16 weeks, more preferably for at least 24 weeks.

Administering the liquid pharmaceutical composition to the human subject by inhalation provides a blood plasma concentration of ensifentrine, which blood plasma concentration of ensifentrine has: a mean Cₘₐₓ of from about 400 pg/mL to about 720 pg/mL; and/or a mean AUC₀₋ₜₐᵤ of from about 2000 pg/mL*h to about 3000 pg/mL*h; and/or a mean Tₘₐₓ at from about 0.8 hours to about 1.3 hours. The mean pharmacokinetic profile (or the pharmacokinetic profile achieved in an individual human subject) may be as further defined above for the liquid pharmaceutical composition.

Administering the liquid pharmaceutical composition to the human subject by inhalation typically provides a mean increase in baseline FEV₁ of at least 20 mL.

The invention also provides a liquid pharmaceutical composition as defined herein for use in a method of treating COPD as defined herein. Further provided is use of a liquid pharmaceutical composition as defined herein in the manufacture of a medicament for use in a method of treating COPD as defined herein.

The invention is described in more detail by the following Example.

### Example

Study Design

A clinical study was conducted to determine the efficacy of ensifentrine in treating COPD compared with placebo. Ensifentrine was administered by nebuliser at a dose of 3 mg twice daily (BID) for 24 weeks. The study was a multi-centre, randomized, double-blind, parallel-group, placebo-controlled trial with around 800 patients and 5:3 randomization.

The study population included patients aged 40-80 years with moderate to severe COPD (FEV₁ 30%-70% p.n., FEV₁/forced vital capacity (FVC) ratio <0.7, with mMRC 2). The randomization stratified (a) the use of stable background maintenance LAMA or LABA therapy use (approx. 50%. yes or no) and (b) cigarette smoking (current or former). Inhaled corticosteroid (ICS) maintenance therapy was permitted in up to 20% of patients under certain provisions.

The primary endpoint of the study was change from baseline in average FEV₁ area under the curve (AUC)0-12 h post-dose at week 12. Secondary endpoints of the study included: peak FEV₁ over 4 hours post-dose at Week 12; morning trough FEV₁ at Week 12; and other endpoints including moderate/severe COPD exacerbations frequency over 24 Weeks.

Methods

Baseline FEV₁ is the mean of the two measurements taken before study medication on the day of first dosing, i.e. <40 minutes and just prior to dosing, both pre-dose on day 1.

Average FEV₁ AUC0-12 h is defined as area under the curve over 12 hours of the FEV₁, divided by 12 hours.

Plasma concentrations of ensifentrine for the assessment of pharmacokinetic (PK) parameters were measured using validated bioanalytical methods for ensifentrine in human plasma with LLOQ of 5 pg/mL.

Samples were taken on the following schedule:
Week 6: 1.0 h (±0.5 h) and 2.5 h (±0.5 h) (even sites) or 1.5 h and 4 h (±1 h) (odd sites);
Week 12: pre-dose (−0.5 h), 4 to 6 h, and 8 to 12 h (even sites) or 0.5 h (±0.25 h), 3 h (±0.5 h), and 6 to 8 h (odd sites);
Week 24: pre-dose (−0.5 h) and 1.5 h (even sites) or pre-dose (−0.5 h) and 1 h (±0.5 h) (odd sites)

Formulation

The investigational product and placebo were provided in 2.5 mL unit dose format in an ampule and administered via a nebuliser. The formulation of the investigational product (ensifentrine suspension formulation comprising crystalline polymorph Form I of ensifentrine) and placebo are shown in Table 1 below.

TABLE 1

| Constituent | Concentration (mg/mL) |
|---|---|
| Ensifentrine particles (RPL554) | 1.2 (for the active) or |
| Dv10 = 0.5 μm | 0 (for Placebo) |
| Dv50 = 1.5 μm | |
| Dv90 = 3.6 μm | |
| Polysorbate 20 (Tween 20) | 0.50 |
| Sorbitan Monolaurate (Span 20) | 0.05 |
| Sodium Dihydrogen Phosphate Dihydrate | 0.744 |
| Disodium Hydrogen Phosphate Dihydrate | 0.853 |
| Sodium Chloride | 8.60 |
| Water | q.s. to 1 mL |

Results

The primary endpoint of average FEV₁ (AUC)0-12 h at Week 12 was met. All subgroups showed improvement in lung function with ensifentrine that was statistically significant. The results are shown in Table 2.

TABLE 2

| Average FEV₁ (AUC)0-12 h | Ensifentrine | | |
|---|---|---|---|
| post-dose at Week 12: | 3 mg | | Placebo |
| Change from Baseline (CFB) | (n = 498) | | (n = 291) |
| Mean Baseline FEV₁, L | 1.3323 | | 1.2316 |
| CFB Least Squares Mean, | 48.2 | (30.0, 66.5) | −45.7 (−69.7, −21.6) |
| mL (95% CI) | | | |
| Placebo-corrected CFB | 93.9 | (64.5, 123.3) | — |
| LS Mean Diff., mL | | | |
| (95% CI) | | | |

| | | |
|---|---|---|
| p-value | <0.0001 | — |

Based on the samples taken during the trial, PK data was modelled for COPD patients with normal renal function. The final popPK model was used to predict parameter estimates in a virtual population of 50000 subjects (per covariate condition) for simulation of ensifentrine PK following nebulization of 3 mg in a population reflecting the population in the trial with normal renal function or mild renal impairment, or with moderate renal impairment. Uncertainty in fixed effects and covariance matrix parameters as well as residual variability were included in the simulations. The parameter uncertainty was accounted for by random resampling (without replacement) of 500 sets of parameter estimates from the bootstrap (750 replicates) in R to create 500 NONMEM simulation control streams. From each set of parameter estimates, the PK profiles for 100 subjects (for each covariate condition) were simulated with IV and IOV from the covariance matrix and residual error in NONMEM. Individual subject concentrations were simulated using NONMEM version 7.4.3, and PK parameters were calculated using R version 3.4.0.

The results are shown in Table 3.

TABLE 3

| Parameter (units) | Median (90% prediction interval) |
|---|---|
| Cₘₐₓ (pg/mL) | 522 (173-1330) |
| AUC₀₋ₜₐᵤ (pg/mL * h ) | 2450 (882-7500) |
| tₘₐₓ (hours) | 1.0 |

### CONCLUSION

It has been found that ensifentrine provides a statistically significant improvement in lung function in all subgroups of COPD patients in the study.

A mean Cₘₐₓ of 522 pg/mL and a mean AUC of 2450 pg/mL*h were achieved in patients with COPD. These values were lower than the mean Cₘₐₓ (852 pg/mL) and mean AUC (mean AUC_{0-∞} of 5929 pg/mL*h) observed following administration of the composition to healthy patients, indicating lower systemic exposure of ensifentrine in COPD patients than in healthy patients.

1. A method of treating chronic obstructive pulmonary disease (COPD) in a human subject, the method comprising administering to the human subject by inhalation a liquid pharmaceutical composition comprising a therapeutically effective dose of ensifentrine or a pharmaceutically acceptable salt thereof,
wherein the liquid pharmaceutical composition provides a blood plasma concentration of ensifentrine after administration by inhalation to the human subject, the blood plasma concentration of ensifentrine comprising one or more of:
a mean Cₘₐₓ of from about 400 pg/mL to about 720 pg/mL;
a mean AUC₀₋ₜₐᵤ of from about 2000 pg/mL*h to about 3000 pg/mL*h; and
a mean Tₘₐₓ of from about 0.6 hours to about 1.5 hours.

2. The method of claim 1, wherein the blood plasma concentration of ensifentrine comprises a mean Cₘₐₓ of from about 500 pg/mL to about 600 pg/mL.

3. The method of claim 1, wherein the blood plasma concentration of ensifentrine comprises a mean AUC₀₋ₜₐᵤ of from about 2300 pg/mL*h to about 2600 pg/mL*h.

4. The method of claim 1, wherein the blood plasma concentration of ensifentrine comprises a mean Tₘₐₓ of from about 0.8 hours to about 1.3 hours.

5. The method of claim 1, wherein the blood plasma concentration of ensifentrine comprises:
a mean Cₘₐₓ of from about 500 pg/mL to about 600 pg/mL; and
a mean AUC₀₋ₜₐᵤ of from about 2350 pg/mL*h to about 2550 pg/mL*h; and
a mean Tₘₐₓ of from about 0.8 hours to about 1.3 hours.

6. The method of claim 1, wherein the mean Cₘₐₓ, the mean AUC₀₋ₜₐᵤ, and the mean Tₘₐₓ are measured at intervals,
wherein the intervals comprise two or more of 1.0 h (±0.5 h), 1.5 h (±0.5 h), 2.5 h (±0.5 h), 3.0 h (±0.5 h), 4.0 h (±1 h), 6.0 h (±1 h), 8.0 h (±1 h), 10.0 h (±1 h), 12.0 h (±1 h), 24.0 h (±1 h), 36.0 h (±1 h), 48.0 h (±1 h), 56.0 h (±1 h) and 60.0 h (±1 h) after the administering to the human subject.

7. The method of claim 1, wherein the blood plasma concentration of ensifentrine is determined using an analytical method having a lower limit of quantitation (LLOQ) of no greater than 5.0 pg/mL.

8. The method of claim 1, wherein the COPD is moderate COPD.

9. The method of claim 1, wherein a renal function of the human subject comprises normal renal function to mild renal impairment.

10. The method of claim 1, wherein the administering to the human subject provides a mean increase in baseline FEV₁ of at least 20 mL.

11. The method of claim 1, wherein the therapeutically effective dose comprises about 2 mg to about 4 mg of ensifentrine or the pharmaceutically acceptable salt thereof.

12. The method of claim 11, wherein the therapeutically effective dose comprises about 3 mg of ensifentrine or the pharmaceutically acceptable salt thereof.

13. The method of claim 1, wherein ensifentrine is in the form of ensifentrine free base.

14. The method of claim 1, wherein the liquid pharmaceutical composition comprises (a) a suspension of ensifentrine particles comprising the ensifentrine or the pharmaceutically acceptable salt thereof and (b) a diluent.

15. The method of claim 14, wherein the ensifentrine particles comprise at least 95 weight percent of the ensifentrine or the pharmaceutically acceptable salt thereof.

16. The method of claim 14, wherein the ensifentrine particles comprise at least 95 weight percent of ensifentrine free base.

17. The method of claim 14, wherein the liquid pharmaceutical composition comprises:
(a) ensifentrine particles at a concentration of from about 1 mg/mL to about 1.4 mg/mL;
(b) a buffer at a concentration of from about 1 mg/mL to about 2 mg/mL;
(c) one or more surfactants at a total concentration of from about 0.3 mg/mL to about 0.8 mg/mL;
(d) a tonicity adjuster at a concentration of from about 5 mg/mL to about 10 mg/mL,
wherein a total weight of ensifentrine or the pharmaceutically acceptable salt thereof in the liquid pharmaceutical composition is from about 2.7 mg to about 3.3 mg.

18. The method of claim 17, wherein the liquid pharmaceutical composition comprises:
(a) ensifentrine particles at a concentration of from about 1 mg/mL to about 1.4 mg/mL;
(b) polysorbate 20 (Tween 20) at a concentration of from about 0.3 mg/mL to about 0.7 mg/mL;
(c) sorbitan monolaurate (Span 20) at a concentration of from 0 mg/mL to about 0.1 mg/mL;
(d) sodium dihydrogen phosphate dihydrate at a concentration of from about 0.5 mg/mL to about 1 mg/mL;
(e) disodium hydrogen phosphate dihydrate at a concentration of from about 0.5 mg/mL to about 1 mg/mL; and
(f) sodium chloride at a concentration of from about 5 mg/mL to about 10 mg/mL,
wherein the total weight of ensifentrine or the pharmaceutically acceptable salt thereof in the liquid pharmaceutical composition is from about 2.7 mg to about 3.3 mg.

19. The method of claim 18, wherein the liquid pharmaceutical composition comprises:
(a) ensifentrine particles at a concentration of 1.2 mg/mL;
(b) polysorbate 20 (Tween 20) at a concentration of 0.5 mg/mL;
(c) sorbitan monolaurate (Span 20) at a concentration of 0.05 mg/mL;
(d) sodium dihydrogen phosphate dihydrate at a concentration of 0.744 mg/mL;
(e) disodium hydrogen phosphate dihydrate at a concentration of 0.853 mg/mL; and
(f) sodium chloride at a concentration of 8.6 mg/mL,
wherein the total weight of ensifentrine or the pharmaceutically acceptable salt thereof in the liquid pharmaceutical composition is 3 mg.

20. The method of claim 14, wherein the ensifentrine particles have a Dv10 of from about 0.3 μm to about 0.9 μm.

21. The method of claim 14, wherein the ensifentrine particles have a Dv50 of from about 0.2 μm to about 5 μm.

22. The method of claim 14, wherein the ensifentrine particles have a Dv90 of from about 2 μm to about 4.5 μm.

23. The method of claim 1, wherein the liquid pharmaceutical composition is administered to the human subject by inhalation from a nebulizer.

24. The method of claim 1, wherein the administering to the human subject is once per day, twice per day, or three times per day.

25. The method of claim 24, wherein the administering to the human subject is twice per day in a first dose and a second dose.

26. The method of claim 25, wherein the first dose is administered to the human subject in the morning and the second dose is administered to the human subject in the evening.

27. The method of claim 25, wherein the first dose is administered within three hours after the human subject waking, and the second dose is administered within three hours before the human subject sleeps.

28. The method of claim 25, wherein the first dose and the second dose are administered about 10 hours to about 14 hours apart.
